(19) United States
(12) Patent Application Publication
(10) Pub. No.: US 2009/0317343 A1
(43) Pub. Date: Dec. 24, 2009

(54) Personal Care Compositions Containing Silicone Elastomer Gels

(76) Inventors: Shaow Lin, Midland, MI (US); Isabella Van Reeth, Shanghai (CN)

Correspondence Address: DOW CORNING CORPORATION CO1232, 2200 W. SALZBURG ROAD, P.O. BOX 994, MIDLAND, MI, 48686-0994, US

(21) Appl. No.: 12/520,579

(22) PCT Filed: Dec. 19, 2007
(86) PCT No.: PCT/US2007/025968

§ 371 Date: Jun. 22, 2009

Related U.S. Application Data
(60) Provisional application No. 60/878,012, filed on Dec. 29, 2006.

(51) Int. Cl. A61K 8/89; A61Q 1/06; A61Q 15/00; A61Q 5/12; A61K 38/43; A61K 8/97; A61Q 5/02; A61K 38/02; A61K 31/07; A61K 47/34; A61Q 1/02; A61K 8/02; A61K 8/04; A61Q 13/00

(52) U.S. Cl. 424/59; 424/63; 424/64; 424/65; 424/70.12; 424/94.1; 424/725; 510/122; 514/2; 514/725; 514/772.3; 512/4

(57) ABSTRACT

Personal care compositions are disclosed containing a silicone elastomer from the reaction of an organohydrogen-siloxane having at least two SiH containing cyclosiloxane rings in its molecule, a compound having at least two aliphatic unsaturated groups in its molecule, and a hydrosilylation catalyst.

### CROSS REFERENCE TO RELATED APPLICATIONS

This application claims priority to U.S. patent application Ser. NO. 60/878,012, filed on Dec. 29, 2006.

### TECHNICAL FIELD

This invention relates to personal care compositions containing a silicone elastomer from the reaction of an organohydrogensiloxane having at least two SiH containing cyclosiloxane rings in its molecule, a compound having at least two aliphatic unsaturated groups in its molecule, and a hydrosilylation catalyst.

### BACKGROUND

Silicone elastomers derived from cyclic organohydrogensiloxanes have been described providing improved gelled compositions. In particular, these silicone elastomers were found to be very efficient at gelling volatile silicone and organic solvents. These silicone elastomers and gels therefrom were disclosed in U.S. patent application Ser. Nos. 60/784340 (filed Mar. 21, 2006), 60/838803 (filed Aug. 18, 2006), 60/799864 (filed May 12, 2006), 60/838802 (filed Aug. 18, 2006), 60/849397 (filed Oct. 4, 2006), and 60/874203 (filed Dec. 11, 2006), all of which are incorporated by reference in their entirety herein. The present invention describes personal care compositions containing the referenced silicone elastomer gels. The silicone elastomer gels have improved aesthetics upon application on skin.

### SUMMARY

This invention relates to a personal care composition comprising a silicone elastomer gel containing a silicone elastomer derived from;
A) an organohydrogensiloxane having at least two SiH containing cyclosiloxane rings in its molecule
B) a compound having at least two aliphatic unsaturated groups in its molecule,
C) a hydrosilylation catalyst,
D) an optional carrier fluid;
E) an optional personal care or healthcare active.

### DETAILED DESCRIPTION

### (A) The Organohydrogensiloxane Having at Least Two SiH Containing Cyclosiloxane Rings

Component (A) in the present invention is an organohydrogensiloxane having at least two SiH containing cyclosiloxane rings in its molecule. Organohydrogensiloxanes suitable as component A) in the present invention are any organopolysiloxanes having in its molecule at least two cyclosiloxane rings with at least one silicon bonded hydrogen (SiH) unit on each siloxane ring. Organopolysiloxanes are well known in the art and are often designated as comprising any number of (R₃SiO_{0.5}), (R₂SiO), (RSiO_{1.5}), or (SiO₂) siloxy units where R is independently any organic group. When R is methyl in the siloxy unit formulas of an organopolysiloxane, the respective siloxy units are often designated as M, D, T or Q siloxy units. Cyclosiloxane rings contain at least three siloxy units (that is the minimum needed in order to form a siloxane ring), and may be any combination of (R₃SiO_{0.5}), (R₂SiO), (RSiO_{1.5}), or (SiO₂) siloxy units that forms a cyclic structure, providing at least one of the cyclic siloxy units on each siloxane ring contains one SiH unit, that is there is at least one (R₂HSiO_{0.5}), (RHSiO), or a (HSiO_{1.5}) siloxy unit present in the ring. These siloxy units can be represented as M^{H}, D^{H}, and T^{H} siloxy units respectively when R is methyl.

The cyclosiloxane rings of A) the organohydrogensiloxane are linked together by a divalent organic or siloxane group, or combination thereof. The divalent linking group may be designated as Y and the cyclosiloxane as G. Thus, the organohydrogensiloxane of the present invention may be represented by the general formula G-[Y-G]ₐ, where G is a cyclosiloxane as described above and Y is a divalent organic, a siloxane, a polyoxyalkylene group, or combination thereof, and the subscript a is greater than zero.

When Y is a divalent organic, it may be a divalent hydrocarbon containing 1 to 30 carbons, either as aliphatic or aromatic structures, and may be branched or un-branched. Alternatively, Y can be an alkylene group containing 2 to 20 carbons, or alternatively containing 4 to 12 carbons.

When Y is a divalent organic, it may also be selected from an organic polymer, such as a polyoxyalkylene group.

When Y is a siloxane group it may be selected from any organopolysiloxane containing at least two divalent hydrocarbon groups, designated as R¹. Thus, the siloxane linking group can be any organopolysiloxane comprising at least two siloxane units represented by the average formula R¹RₘSiO_{(4-m)/2}

wherein
R is an organic group,
R¹ is a divalent hydrocarbon, and
m is zero to 3
   The R¹ group may be present on any mono, di, or tri-siloxy unit in an organopolysiloxane molecule, for example; (R¹R₂SiO_{0.5}), (R¹RSiO), or (R¹SiO_{1.5}), as well as in combination with other siloxy units not containing an R¹ substituent, such as (R₃SiO_{0.5}), (R₂SiO), (RSiO_{1.5}), or (SiO₂) siloxy units where R is independently any organic group providing there are at least two R¹ substituents in the organopolysiloxane. Representative R¹ groups include; ethylene, propylene, butylene, isobutylene, hexylene, and similar homologs. Alternatively, R¹ is ethylene.

Representative, non-limiting, examples of such siloxane based structures suitable as siloxane linking groups include;

(R₂R¹SiO_{0.5})(R₂SiO)ₓ(R₂R¹SiO_{0.5})

(R₃SiO_{0.5})(R₂SiO)ₓ(R¹RSiO)_{y}(R₃SiO_{0.5})

(R₃SiO_{0.5})(R₂SiO)ₓ(R¹RSiO)_{y}(RSiO_{1.5})_{z}(R₃SiO_{0.5})
where x≧0, y≧2, and z is ≧0

Organohydrogensiloxane having at least two Sill containing cyclosiloxane rings (component A) may be prepared via a hydrosilylation reaction of
a) an organohydrogencyclosiloxane having at least two Sill units on the siloxane ring and,
B) a compound containing at least two aliphatic unsaturated groups in its molecule. The organohydrogencyclosiloxane (a) having at least two SiH units on the siloxane ring may contain any number of siloxy units (as defined above) provided there are at least two SiH units on the cyclosiloxane ring. For example, the cyclic siloxane can comprise any number of M, M^{H}, D, D^{H}, or T^{H} siloxy units. Representative, non-limiting examples of such organohydrogencyclosiloxanes useful to prepare component (A) have the average formula D^{H}ₐD_{b} where a is ≧1 and b is ≧0, and a+b≧3. Alternatively, the organohydrogencyclosiloxane may be D^{H}₄, D^{H}₅, D^{H}₆, or mixtures thereof.

Suitable compounds containing at least two aliphatic unsaturated groups in its molecule are described below as component B).

Hydrosilylation reactions involving organohydrogensiloxanes and unsaturated compounds are well known. Any suitable hydrosilylation catalysts know in the art may be used, or alternatively may be selected from those described below as component C). Any of the known hydrosilylation techniques and reactions may be employed to prepare component A) from i) organohydrogencyclosiloxane having at least two SiH units on the siloxane ring and, ii) a compound containing at least two aliphatic unsaturated groups in its molecule. However, the reaction is conducted in such a manner to provide an organohydrogensiloxane having at least two SiH containing cyclosiloxane rings in its molecule.

Thus, component A of the present invention contains at least two silicon-bonded hydrogen atom per molecule, alternatively at least 4 silicon-bonded hydrogen atoms per molecule, or alternatively at least 6 silicon-bonded hydrogen atoms per molecule. This can be accomplished by using in the hydrosilylation reaction a molar excess of the organohydrogencyclosiloxane vs the compound containing at least two aliphatic unsaturated groups in its molecule. The molar excess may be expressed as the molar ratio of SiH units to unsaturated group, such ratio may range from 2/1 to 8/1, alternatively from 2/1 to 6/1, or alternatively from 3/1 to 4/1.

Alternatively, the organohydrogensiloxane useful as component A) may be selected from any of the organohydrogensiloxanes taught in WO03/093349, which is herein incorporated by reference for its teaching of suitable organohydrogensiloxanes.

The organohydrogensiloxane useful as component A) in the present invention typically have a viscosity from 5 to 50,000 mPa·s, alternatively from 10 to 10,000 mPa·s, or alternatively from 25 to 2,000 mPa·s.

Representative, non-limiting examples of component A) include;

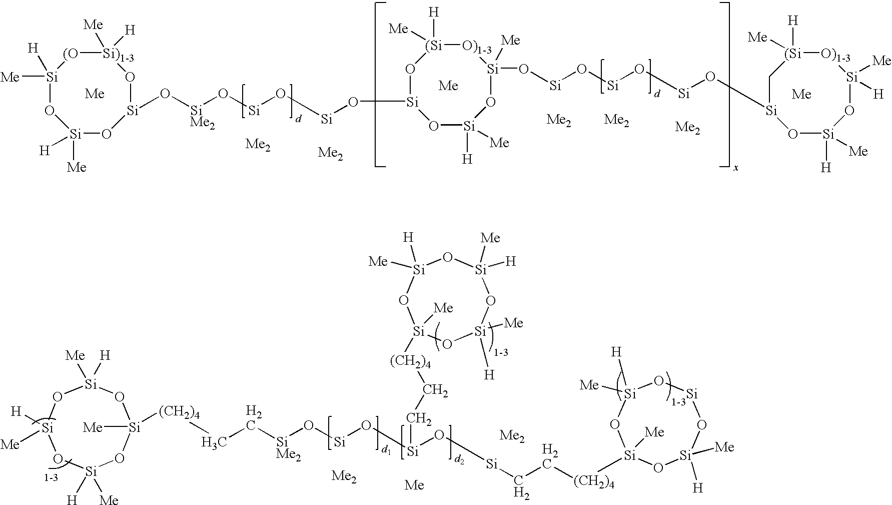

Additives known as inhibitors or stabilizers may be added to component A). Inhibitors such as those described in WO 03/093369 may be added for the purpose of stabilizing component A) during storage, or prior to the addition of component B) to prepare the silicone elastomer gel. The inhibitor may be selected from any compound known to have inhibiting effects of platinum based hydrosilylation reactions. A particularly preferred inhibitor is vitamin A palmitate, or VAP. When VAP is used, it is typically added at 0.05 to 2.0 parts per 100 parts of component A).

### (B) The Compound Having at Least Two Aliphatic Unsaturated Groups in Its Molecule

Component (B) is a compound containing at least two aliphatic unsaturated groups in its molecule. The compound may be any diene, diyne or ene-yne compound. Diene, diyne or ene-yne compounds are those compounds (including polymeric compounds) wherein there are at least two aliphatic unsaturated groups with some separation between the groups within the molecule. Typically, the unsaturation groups are at the termini of the compound, or pendant if part of a polymeric compound. Compounds containing terminal or pendant unsaturated groups can be represented by the formula R²—Y—R² where R² is an monovalent unsaturated aliphatic group and Y is a divalent organic or siloxane group or a combination of these. Typically R² is CH₂═CH—, CH₂═CHCH₂—, CH₂═C(CH₃)CH₂— or CH≡C—, and similar substituted unsaturated groups such as H₂C═C(CH₃)—, and HC≡(CH₃)—.

The compound may be considered as being a “hydrocarbon”, “organic polymer”, or “siloxane”, or combinations thereof, depending on the selection of Y.

In one embodiment, the component (B) is selected from a compound having the formula R²—Y—R² where R² is a monovalent unsaturated aliphatic group and Y is a divalent hydrocarbon, herein denoted as (B¹). The divalent hydrocarbon may contain 1 to 30 carbons, either as aliphatic or aromatic structures, and may be branched or un-branched. Component (B¹) may be exemplified by, but not limited to 1,4-pentadiene, 1,5-hexadiene; 1,6-heptadiene; 1,7-octadiene, 1,8-nonadiene, 1,9-decadiene, 1,11-dodecadiene, 1,13-tetradecadiene, and 1,19-eicosadiene, 1,3-butadiyne, 1,5-hexadiyne (dipropargyl), and 1-hexene-5-yne.

In another embodiment, the component (B) is selected from a R²—Y—R² compound where Y is a siloxane, herein denoted as (B²). When Y is a siloxane group it may be selected from any organopolysiloxane bonded to at least two organic groups having aliphatic unsaturation, designated as R², to form R²—Y—R² structures. Thus, component (B²) can be any organopolysiloxane comprising at least two siloxane units represented by the average formula R²RₘSiO_{(4-m)/2}

wherein
R is an organic group,
R² is a monovalent unsaturated aliphatic group as defined above, and
m is zero to 3

The R² group may be present on any mono, di, or tri siloxy unit in an organopolysiloxane molecule, for example; (R²R₂SiO_{0.5}), (R²RSiO), or (R²SiO_{0.5}); as well as in combination with other siloxy units not containing an R² substituent, such as (R₃SiO_{0.5}), (R₂SiO), (RSiO_{1.5}), or (SiO₂) siloxy units where R is independently any organic group; providing there are at least two R² substituents in the organopolysiloxane.

Representative, non-limiting, examples of such siloxane based R²—Y—R² structures suitable as component (B²) include;

(R₂R²SiO_{0.5})(SiO₂)_{w}(R₂R²SiO_{0.5})

(R₂R²SiO_{0.5})(SiO₂)_{w}(R₂SiO)ₓ(R₂R²SiO_{0.5})

(R₂R²SiO_{0.5})(R₂SiO)ₓ(R₂R²SiO_{0.5})

(R₃SiO_{0.5})(R₂SiO)ₓ(R²RSiO)_{y}(R₃SiO_{0.5})

(R₃SiO_{0.5})(R₂SiO)ₓ(R²RSiO)_{y}(RSiO_{1.5})_{z}(R₃SiO_{0.5})

(R₃SiO_{0.5})(R₂SiO)ₓ(R²RSiO)_{y}(SiO₂)_{w}(R₃SiO_{0.5})
where w≧0, x≧0, y≧2, and z is ≧b 0.

In another embodiment, component (B) is selected from a R²—Y—R² compound, herein denoted as (B³), where R² is as defined above and Y is a polyoxyalkylene group having the formula (CₙH₂ₙO)b wherein n is from 2 to 4 inclusive,
b is greater than 2,
   alternatively b can range from 2 to 100,
      or alternatively b can range from 2 to 50.
         The polyoxyalkylene group typically can comprise oxyethylene units (C₂H₄O), oxypropylene units (C₃H₆O), oxybutylene units (C₄H₈O), or mixtures thereof. Thus, the R²—Y—R² compound may be selected from a polyoxyalkylene group having the formula R²—[(C₂H₄O)_{c} (C₃H₆O)_{d} (C₄H₈O)ₑ]—R² where c, d, and e may each independently range from 0 to 100, providing the sum of c+d+e is greater than 2, alternatively the sum of c+d+e ranges from 2 to 100, or alternatively the sum of c+d+e ranges from 2 to 50.

Alternatively, the polyoxyalkylene group comprises only oxypropylene units (C3H6O)_{d}. Representative, non-limiting examples of polyoxypropylene containing R²—Y—R² compounds include;

H₂C═CHCH₂[C₃H₆O]_{d}CH₂CH═CH₂

H₂C═CH[C₃H₆O]_{d}CH═CH₂

H₂C═C(CH₃)CH₂[C₃H₆O]_{d}CH₂C(CH₃)═CH₂

HC≡CCH₂[C₃H₆O]_{d}CH₂C≡CH

HC≡CC(CH₃)₂[C₃H₆O]_{d}C(CH₃)₂C≡CH

where d is as defined above.
Representative, non-limiting examples of polyoxybutylene containing R²—Y—R² compounds include;

H₂C═CHCH₂[C₄H₈O]ₑCH₂CH═CH₂

H₂C═CH[C₄H₈O]ₑCH═CH₂

H₂C═C(CH₃)CH₂[C₄H₈O]ₑCH₂C(CH₃)═CH₂

HC≡CCH₂[C₄H₈O]ₑCH₂C≡CH

HC≡CC(CH₃)₂[C₄H₈O]ₑC(CH₃)₂C≡CH

In another embodiment, component (B) is selected from a R²—Y—R² compound, herein denoted as (B⁴), where R² is as defined above and Y is a polyalkylene group, selected from C2 to C6 alkylene units or their isomers. One example is polyisobutylene group which is a polymer containing isobutylene unit.

The molecular weight of the polyisobutylene group may vary, but typically ranges from 100 to 10,000 g/mole. Representative, non-limiting examples of R²—Y—R² compounds containing a polyisobutylene group includes those commercially available from BASF under the tradename of OPPONOL BV, such as OPPONOL BV 5K, a diallyl terminated polyisobutylene having an average molecular weight of 5000 g/mole.

In yet another embodiment, component (B) is selected from a R²—Y—R² compound, herein denoted as (B⁵), where R² is as defined above and Y is a hydrocarbon-silicone copolymer group. The hydrocarbon-silicone copolymer group may have the formula

—[R¹ᵤ(R₂SiO)ᵥ]ₘ—

where R¹ and R are as defined above;
u and v are independently ≧1, alternatively u ranges from 1 to 20, alternatively v ranges from 2 to 500, or from 2 to 200,
m is >1, alternatively m ranges from 2 to 500, alternatively m ranges from 2 to 100.
   R²—Y—R² compounds having a hydrocarbon-silicone copolymer group may be prepared via a hydrosilylation reaction between an α-ω unsaturated hydrocarbon, such as those described above as B¹, and an organohydrogensiloxane. A representative, non-limiting example of such a reaction is shown below.

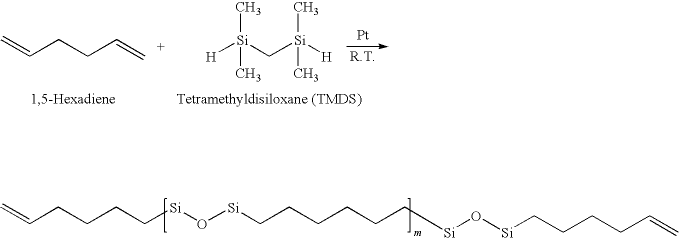

Component (B) may also be a mixture of any diene, diyne or ene-yne compound, such as combinations of B¹, B² , B³ , B⁴ , and B⁵.

The amounts of component (A) and component (B) used to prepare the present composition will depend on the individual components and the desired SiH to aliphatic unsaturation ratio. The ratio of SiH in component (A) to aliphatic unsaturation from component (B) useful to prepare the compositions of the present invention can be from 10:1 to 1:10, alternatively 5:1 to 1:5, or alternatively 4:1 to 1:4.

If components (A) and (B) are not the only materials containing aliphatic unsaturated groups and SiH-containing groups in the present composition, then the above ratios relate to the total amount of such groups present in the composition rather than only those components.

### (C) The Hydrosilylation Catalyst

Component (C) comprises any catalyst typically employed for hydrosilylation reactions. It is preferred to use platinum group metal-containing catalysts. By platinum group it is meant ruthenium, rhodium, palladium, osmium, iridium and platinum and complexes thereof. Platinum group metal-containing catalysts useful in preparing the compositions of the present invention are the platinum complexes prepared as described by Willing, U. S. Pat. No. 3,419,593, and Brown et al, U. S. Pat. No. 5,175,325, each of which is hereby incorporated by reference to show such complexes and their preparation. Other examples of useful platinum group metal-containing catalysts can be found in Lee et al., U.S. Pat. No. 3,989,668; Chang et al., U.S. Pat. No. 5,036,117; Ashby, U.S. Pat. No. 3,159,601; Lamoreaux, U.S. Pat. No. 3,220,972; Chalk et al., U.S. Pat. No. 3,296,291; Modic, U.S. Pat. No. 3,516,946; Karstedt, U. S. Pat. No. 3,814,730; and Chandra et al., U.S. Pat. No. 3,928,629 all of which are hereby incorporated by reference to show useful platinum group metal-containing catalysts and methods for their preparation. The platinum-containing catalyst can be platinum metal, platinum metal deposited on a carrier such as silica gel or powdered charcoal, or a compound or complex of a platinum group metal. Preferred platinum-containing catalysts include chloroplatinic acid, either in hexahydrate form or anhydrous form, and or a platinum-containing catalyst which is obtained by a method comprising reacting chloroplatinic acid with an aliphatically unsaturated organosilicon compound such as divinyltetramethyldisiloxane, or alkene-platinum-silyl complexes as described in U.S. patent application Ser. No. 10/017229, filed Dec. 7, 2001, such as (COD)Pt(SiMeCl₂)₂, where COD is 1,5-cyclooctadiene and Me is methyl. These alkeneplatinum-silyl complexes may be prepared, for example by mixing 0.015 mole (COD)PtCl₂ with 0.045 mole COD and 0.0612 moles HMeSiCl₂.

The appropriate amount of the catalyst will depend upon the particular catalyst used. The platinum catalyst should be present in an amount sufficient to provide at least 2 parts per million (ppm), preferably 4 to 200 ppm of platinum based on total weight percent solids (all non-solvent ingredients) in the composition. It is highly preferred that the platinum is present in an amount sufficient to provide 4 to 150 weight ppm of platinum on the same basis. The catalyst may be added as a single species or as a mixture of two or more different species.

### (D) The Carrier Fluid

The silicone elastomers may be contained in an optional carrier fluid (D). Although it is not required, typically the carrier fluid may be the same as the solvent used for conducting the hydrosilylation reaction as described above. Suitable carrier fluids include silicones, both linear and cyclic, organic oils, organic solvents and mixtures of these. Specific examples of solvents may be found in U.S. Pat. No. 6,200,581, which is hereby incorporated by reference for this purpose.

Typically, the carrier fluid is a low viscosity silicone or a volatile methyl siloxane or a volatile ethyl siloxane or a volatile methyl ethyl siloxane having a viscosity at 25° C. in the range of 1 to 1,000 mm²/sec such as hexamethylcyclotrisiloxane, octamethylcyclotetrasiloxane, decamethylcyclopentasiloxane, dodecamethylcyclohexasiloxane, octamethyltrisiloxane, decamethyltetrasiloxane, dodecamethylpentasiloxane, tetradecamethylhexasiloxane, hexadeamethylheptasiloxane, heptamethyl-3-{(trimethylsilyl)oxy)}trisiloxane, hexamethyl-3,3,bis{(trimethylsilyl)oxy}trisiloxane pentamethyl{(trimethylsilyl)oxy}cyclotrisiloxane as well as polydimethylsiloxanes, polyethylsiloxanes, polymethylethylsiloxanes, polymethylphenylsiloxanes, polydiphenylsiloxanes.

Organic solvents may be exemplified by, but not limited to, aromatic hydrocarbons, aliphatic hydrocarbons, alcohols, aldehydes, ketones, amines, esters, ethers, glycols, glycol ethers, alkyl halides and aromatic halides. Hydrocarbons including isododecane, isohexadecane, Isopar L (C11-C13), Isopar H (C11-C12), hydrogentated polydecen. Ethers and esters including isodecyl neopentanoate, neopentylglycol heptanoate, glycol distearate, dicaprylyl carbonate, diethylhexyl carbonate, propylene glycol n butyl ether, ethyl-3 ethoxypropionate, propylene glycol methyl ether acetate, tridecyl neopentanoate, propylene glycol methylether acetate (PGMEA), propylene glycol methylether (PGME). octyldodecyl neopentanoate, diisobutyl adipate, diisopropyl adipate, propylene glycol dicaprylate/dicaprate, and octyl palmitate. Additional organic carrier fluids suitable as a stand alone compound or as an ingredient to the carrier fluid include fats, oils, fatty acids, and fatty alcohols.

The amount of carrier fluid is such that there is 0 to 98 weight percent, alternatively 0.5 to 80 weight percent, alternatively 5 to 70 weight percent, of carrier fluid in composition containing (A) and (B) and (D), where the sum of (A), (B), and (D) is 100 weight percent.

### E) Personal or Healthcare Active

Component E) is active selected from any personal or health care active. As used herein, a “personal care active” means any compound or mixtures of compounds that are known in the art as additives in the personal care formulations that are typically added for the purpose of treating hair or skin to provide a cosmetic and/or aesthetic benefit. A “healthcare active” means any compound or mixtures of compounds that are known in the art to provide a pharmaceutical or medical benefit. Thus, “healthcare active” include materials consider as an active ingredient or active drug ingredient as generally used and defined by the United States Department of Health & Human Services Food and Drug Administration, contained in Title 21, Chapter I, of the Code of Federal Regulations, Parts 200-299 and Parts 300-499.

Thus, active ingredient can include any component that is intended to furnish pharmacological activity or other direct effect in the diagnosis, cure, mitigation, treatment, or prevention of disease, or to affect the structure or any function of the body of a human or other animals. The phrase can include those components that may undergo chemical change in the manufacture of drug products and be present in drug products in a modified form intended to furnish the specified activity or effect.

Some representative examples of active ingredients include; drugs, vitamins, minerals; hormones; topical antimicrobial agents such as antibiotic active ingredients, antifungal active ingredients for the treatment of athlete's foot, jock itch, or ringworm, and acne active ingredients; astringent active ingredients; deodorant active ingredients; wart remover active ingredients; corn and callus remover active ingredients; pediculicide active ingredients for the treatment of head, pubic (crab), and body lice; active ingredients for the control of dandruff, seborrheic dermatitis, or psoriasis; and sunburn prevention and treatment agents.

Useful active ingredients for use in processes according to the invention include vitamins and its derivatives, including “pro-vitamins”. Vitamins useful herein include, but are not limited to, Vitamin A₁, retinol, C₂-C₁₈ esters of retinol, vitamin E, tocopherol, esters of vitamin E, and mixtures thereof. Retinol includes trans-retinol, 1, 3-cis-retinol, 11-cis-retinol, 9-cis-retinol, and 3,4-didehydro-retinol, Vitamin C and its derivatives, Vitamin B₁, Vitamin B₂, Pro Vitamin B5, panthenol, Vitamin B₆, Vitamin B₁₂, niacin, folic acid, biotin, and pantothenic acid. Other suitable vitamins and the INCI names for the vitamins considered included herein are ascorbyl dipalmitate, ascorbyl methylsilanol pectinate, ascorbyl palmitate, ascorbyl stearate, ascorbyl glucocide, sodium ascorbyl phosphate, sodium ascorbate, disodium ascorbyl sulfate, potassium (ascorbyl/tocopheryl) phosphate.

RETINOL, it should be noted, is an International Nomenclature Cosmetic Ingredient Name (INCI) designated by The Cosmetic, Toiletry, and Fragrance Association (CTFA), Washington DC, for vitamin A. Other suitable vitamins and the INCI names for the vitamins considered included herein are RETINYL ACETATE, RETINYL PALMITATE, RETINYL PROPIONATE, α-TOCOPHEROL, TOCOPHERSOLAN, TOCOPHERYL ACETATE, TOCOPHERYL LINOLEATE, TOCOPHERYL NICOTINATE, and TOCOPHERYL SUCCINATE.

Some examples of commercially available products suitable for use herein are Vitamin A Acetate and Vitamin C, both products of Fluka Chemie AG, Buchs, Switzerland; COVI-OX T-50, a vitamin E product of Henkel Corporation, La Grange, Ill.; COVI-OX T-70, another vitamin E product of Henkel Corporation, La Grange, Ill.; and vitamin E Acetate, a product of Roche Vitamins & Fine Chemicals, Nutley, N.J.

The active ingredient used in processes according to the invention can be an active drug ingredient. Representative examples of some suitable active drug ingredients which can be used are hydrocortisone, ketoprofen, timolol, pilocarpine, adriamycin, mitomycin C, morphine, hydromorphone, diltiazem, theophylline, doxorubicin, daunorubicin, heparin, penicillin G, carbenicillin, cephalothin, cefoxitin, cefotaxime, 5-fluorouracil, cytarabine, 6-azauridine, 6-thioguanine, vinblastine, vincristine, bleomycin sulfate, aurothioglucose, suramin, mebendazole, clonidine, scopolamine, propranolol, phenylpropanolamine hydrochloride, ouabain, atropine, haloperidol, isosorbide, nitroglycerin, ibuprofen, ubiquinones, indomethacin, prostaglandins, naproxen, salbutamol, guanabenz, labetalol, pheniramine, metrifonate, and steroids.

Considered to be included herein as active drug ingredients for purposes of the present invention are antiacne agents such as benzoyl peroxide and tretinoin; antibacterial agents such as chlorohexadiene gluconate; antifungal agents such as miconazole nitrate; anti-inflammatory agents; corticosteroidal drugs; non-steroidal anti-inflammatory agents such as diclofenac; antipsoriasis agents such as clobetasol propionate; anesthetic agents such as lidocaine; antipruritic agents; antidermatitis agents; and agents generally considered barrier films.

The active component E) of the present invention can be a protein, such as an enzyme. The internal inclusion of enzymes in the silicone vesicle have advantages to prevent enzymes from deactivating and maintain bioactive effects of enzymes for longer time. Enzymes include, but are not limited to, commercially available types, improved types, recombinant types, wild types, variants not found in nature, and mixtures thereof. For example, suitable enzymes include hydrolases, cutinases, oxidases, transferases, reductases, hemicellulases, esterases, isomerases, pectinases, lactases, peroxidases, laccases, catalases, and mixtures thereof. Hydrolases include, but are not limited to, proteases (bacterial, fungal, acid, neutral or alkaline), amylases (alpha or beta), lipases, mannanases, cellulases, collagenases, lisozymes, superoxide dismutase, catalase, and mixtures thereof. Said protease include, but are not limited to, trypsin, chymotrypsin, pepsin, pancreatin and other mammalian enzymes; papain, bromelain and other botanical enzymes; subtilisin, epidermin, nisin, naringinase(L-rhammnosidase) urokinase and other bacterial enzymes. Said lipase include, but are not limited to, triacyl-glycerol lipases, monoacyl-glycerol lipases, lipoprotein lipases, e.g. steapsin, erepsin, pepsin, other mammalian, botanical, bacterial lipases and purified ones. Natural papain is preferred as said enzyme. Further, stimulating hormones, e.g. insulin, can be used together with these enzymes to boost the effectiveness of them.

Component E) may also be a sunscreen agent. The sunscreen agent can be selected from any sunscreen agent known in the art to protect skin from the harmful effects of exposure to sunlight. The sunscreen compound is typically chosen from an organic compound, an inorganic compound, or mixtures thereof that absorbs ultraviolet (UV) light. Thus, representative non limiting examples that can be used as the sunscreen agent include; Aminobenzoic Acid, Cinoxate, Diethanolamine Methoxycinnamate, Digalloyl Trioleate, Dioxybenzone, Ethyl 4-[bis(Hydroxypropyl)] Aminobenzoate, Glyceryl Aminobenzoate, Homosalate, Lawsone with Dihydroxyacetone, Menthyl Anthranilate, Octocrylene, Octyl Methoxycinnamate, Octyl Salicylate, Oxybenzone, Padimate O, Phenylbenzimidazole Sulfonic Acid, Red Petrolatum, Sulisobenzone, Titanium Dioxide, and Trolamine Salicylate, cetaminosalol, Allatoin PABA, Benzalphthalide, Benzophenone, Benzophenone 1-12, 3-Benzylidene Camphor, Benzylidenecamphor Hydrolyzed Collagen Sulfonamide, Benzylidene Camphor Sulfonic Acid, Benzyl Salicylate, Bornelone, Bumetriozole, Butyl Methoxydibenzoylmethane, Butyl PABA, Ceria/Silica, Ceria/Silica Talc, Cinoxate, DEA-Methoxycinnamate, Dibenzoxazol Naphthalene, Di-t-Butyl Hydroxybenzylidene Camphor, Digalloyl Trioleate, Diisopropyl Methyl Cinnamate, Dimethyl PABA Ethyl Cetearyldimonium Tosylate, Dioctyl Butamido Triazone, Diphenyl Carbomethoxy Acetoxy Naphthopyran, Disodium Bisethylphenyl Tiamminotriazine Stilbenedisulfonate, Disodium Distyrylbiphenyl Triaminotriazine Stilbenedisulfonate, Disodium Distyrylbiphenyl Disulfonate, Drometrizole, Drometrizole Trisiloxane, Ethyl Dihydroxypropyl PABA, Ethyl Diisopropylcinnamate, Ethyl Methoxycinnamate, Ethyl PABA, Ethyl Urocanate, Etrocrylene Ferulic Acid, Glyceryl Octanoate Dimethoxycinnamate, Glyceryl PABA, Glycol Salicylate, Homosalate, Isoamyl p-Methoxycinnamate, Isopropylbenzyl Salicylate, Isopropyl Dibenzolylmethane, Isopropyl Methoxycinnamate, Menthyl Anthranilate, Menthyl Salicylate, 4-Methylbenzylidene, Camphor, Octocrylene, Octrizole, Octyl Dimethyl PABA, Octyl Methoxycinnamate, Octyl Salicylate, Octyl Triazone, PABA, PEG-25 PABA, Pentyl Dimethyl PABA, Phenylbenzimidazole Sulfonic Acid, Polyacrylamidomethyl Benzylidene Camphor, Potassium Methoxycinnamate, Potassium Phenylbenzimidazole Sulfonate, Red Petrolatum, Sodium Phenylbenzimidazole Sulfonate, Sodium Urocanate, TEA-Phenylbenzimidazole Sulfonate, TEA-Salicylate, Terephthalylidene Dicamphor Sulfonic Acid, Titanium Dioxide, Zinc Dioxide, Serium Dioxide, TriPABA Panthenol, Urocanic Acid, and VA/Crotonates/Methacryloxybenzophenone-1 Copolymer.

These sunscreen agent can be selected one or combination of more than one. Further, the silicone vesicle can contain one sunscreen agent in inner phase, and another in outer phase, e.g. containing oil-soluble sunscreen agent in inner phase and water-dispersible one in outer phase of this silicone vesicle. In this usage, the silicone vesicle is useful to stabilize the combination of different sunscreens for some organic sunscreen agents are colored by contacting with Titanium dioxide directly.

Alternatively, the sunscreen agent is a cinnamate based organic compound, or alternatively, the sunscreen agent is octyl methoxycinnamate, such as Uvinul® MC 80 an ester of para-methoxycinnamic acid and 2-ethylhexanol.

Component E) may also be a fragrance or perfume. The perfume can be any perfume or fragrance active ingredient commonly used in the perfume industry. These compositions typically belong to a variety of chemical classes, as varied as alcohols, aldehydes, ketones, esters, ethers, acetates, nitrites, terpenic hydrocarbons, heterocyclic nitrogen or sulfur containing compounds, as well as essential oils of natural or synthetic origin. Many of these perfume ingredients are described in detail in standard textbook references such as Perfume and Flavour Chemicals, 1969, S. Arctander, Montclair, N.J.

Fragrances may be exemplified by, but not limited to, perfume ketones and perfume aldehydes. Illustrative of the perfume ketones are buccoxime; iso jasmone; methyl beta naphthyl ketone; musk indanone; tonalid/musk plus; Alpha-Damascone, Beta-Damascone, Delta-Damascone, Iso-Damascone, Damascenone, Damarose, Methyl-Dihydrojasmonate, Menthone, Carvone, Camphor, Fenchone, Alpha-Ionone, Beta-Ionone, Gamma-Methyl so-called Ionone, Fleuramone, Dihydrojasmone, Cis-Jasmone, Iso-E-Super, Methyl-Cedrenyl-ketone or Methyl- Cedrylone, Acetophenone, Methyl-Acetophenone, Para-MethoxyAcetophenone, Methyl-Beta-Naphtyl-Ketone, Benzyl-Acetone, Benzophenone, Para-Hydroxy-Phenyl-Butanone, Celery Ketone or Livescone, 6-Isopropyldecahydro-2-naphtone, Dimethyl-Octenone, Freskomenthe, 4-(1-Ethoxyvinyl)-3,3,5,5,-tetramethyl-Cyclohexanone, Methyl-Heptenone, 2-(2-(4-Methyl-3-cyclohexen-1-yl)propyl)-cyclopentanone, 1-(p-Menthen-6(2)-yl)-1-propanone, 4-(4-Hydroxy-3-methoxyphenyl)-2-butanone, 2-Acetyl-3,3-Dimethyl-Norbomane, 6,7-Dihydro-1,1,2,3,3-Pentamethyl-4(5H)-Indanone, 4-Damascol, Dulcinyl or Cassione, Gelsone, Hexalon, Isocyclemone E, Methyl Cyclocitrone, Methyl-Lavender-Ketone, Orivon, Para-tertiary-Butyl-Cyclohexanone, Verdone, Delphone, Muscone, Neobutenone, Plicatone, Veloutone, 2,4,4,7-Tetramethyl-oct-6-en-3-one, and Tetrameran.

More preferably, the perfume ketones are selected for its odor character from Alpha Damascone, Delta Damascone, Iso Damascone, Carvone, Gamma-Methyl-lonone, Iso-E-Super, 2,4,4,7-Tetramethyl-oct-6-en-3-one, Benzyl Acetone, Beta Damascone, Damascenone, methyl dihydrojasmonate, methyl cedrylone, and mixtures thereof.

Preferably, the perfume aldehyde is selected for its odor character from adoxal; anisic aldehyde; cymal; ethyl vanillin; florhydral; helional; heliotropin; hydroxycitronellal; koavone; lauric aldehyde; lyral; methyl nonyl acetaldehyde; P. T. bucinal; phenyl acetaldehyde; undecylenic aldehyde; vanillin; 2,6,10-trimethyl-9-undecenal, 3-dodecen-1-al, alpha-n-amyl cinnamic aldehyde, 4-methoxybenzaldehyde, benzaldehyde, 3-(4-tert butylphenyl)-propanal, 2-methyl-3-(para-methoxyphenyl propanal, 2-methyl-4-(2,6,6-trimethyl-2(1)-cyclohexen-1-y1) butanal, 3-phenyl-2-propenal, cis-/trans-3,7-dimethyl-2,6-octadien-1-al, 3,7-dimethyl-6-octen-1-al, [(3,7-dimethyl-6-octenyl)oxy] acetaldehyde, 4-isopropylbenzyaldehyde, 1,2,3,4,5,6,7,8-octahydro-8,8-dimethyl-2-naphthaldehyde , 2,4-dimethyl-3-cyclohexen-1-carboxaldehyde, 2-methyl-3-(isopropylphenyl)propanal, 1-decanal; decyl aldehyde, 2,6-dimethyl-5-heptenal, 4-(tricyclo[5.2.1.0(2,6)]-decylidene-8)-butanal, octahydro-4,7-methano-1H- indenecarboxaldehyde, 3-ethoxy-4-hydroxy benzaldehyde, para-ethyl-alpha, alpha-dimethyl hydrocinnamaldehyde, alpha-methyl-3,4-(methylenedioxy)-hydrocinnamaldehyde, 3,4-methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, Undecenal, 2,4,6-trimethyl-3-cyclohexene-1-carboxaldehyde, 4-(3)(4-methyl-3-pentenyl)-3-cyclohexen-carboxaldehyde, 1-dodecanal, 2,4-dimethyl cyclohexene-3-carboxaldehyde, 4-(4-hydroxy-4-methyl pentyl)-3-cylohexene-1-carboxaldehyde, 7-methoxy3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, dihydrocinnamic aldehyde, 1-methyl-4-(4-methyl-3-pentenyl)-3-cyclohexene-1-carbox aldehyde, 5 or 6 methoxyl 0 hexahydro-4,7-methanoindan-1 or 2- carboxaldehyde, 3,7-dimethyloctan-1-al, 1 -undecanal, 10-undecen-1-al, 4-hydroxy-3-methoxy benzaldehyde, 1-methyl-3-(4-methylpentyl)-3-cyclhexenecarboxaldehyde, 7-hydroxy-3,7-dimethyl-octanal, trans-4-decenal, 2,6-nonadienal, paratolylacetaldehyde; 4-methylphenylacetaldehyde, 2-methyl-4-(2,6,6-trimethyl-1-cyclohexen-1-yl)-2-butena 1, ortho-methoxycinnamic aldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanoindan-1-carboxaldehyde, 2-methyl octanal, alpha-methyl-4-(1-methyl ethyl) benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, Hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal, methylnonyl acetaldehyde, hexanal, trans-2-hexenal, 1-p-menthene-q-carboxaldehyde and mixtures thereof.

More preferred aldehydes are selected for their odor character from 1-decanal, benzaldehyde, florhydral, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, P.T. Bucinal, lyral, cymal, methyl nonyl acetaldehyde, hexanal, trans-2-hexenal, and mixture thereof.

In the above list of perfume ingredients, some are commercial names conventionally known to one skilled in the art, and also includes isomers. Such isomers are also suitable for use in the present invention.

Component E) may also be one or more plant extract. Examples of these components are as follows: Ashitaba extract, avocado extract, hydrangea extract, Althea extract, Arnica extract, aloe extract, apricot extract, apricot kernel extract, Ginkgo Biloba extract, fennel extract, turmeric[Curcuma] extract, oolong tea extract, rose fruit extract, Echinacea extract, Scutellaria root extract, Phellodendro bark extract, Japanese Coptis extract, Barley extract, Hyperium extract, White Nettle extract, Watercress extract, Orange extract, Dehydrated saltwater, seaweed extract, hydrolyzed elastin, hydrolyzed wheat powder, hydrolyzed silk, Chamomile extract, Carrot extract, Artemisia extract, Glycyrrhiza extract, hibiscustea extract, Pyracantha Fortuneana Fruit extract, Kiwi extract, Cinchona extract, cucumber extract, guanocine, Gardenia extract, Sasa Albo-marginata extract, Sophora root extract, Walnut extract, Grapefruit extract, Clematis extract, Chlorella extract, mulberry extract, Gentiana extract, black tea extract, yeast extract, burdock extract, rice bran ferment extract, rice germ oil, comfrey extract, collagen, cowberry extract, Gardenia extract, Asiasarum Root extract, Family of Bupleurum extract, umbilical cord extract, Salvia extract, Saponaria extract, Bamboo extract, Crataegus fruit extract, Zanthoxylum fruit extract, shiitake extract, Rehmannia root extract, gromwell extract, Perilla extract, linden extract, Filipendula extract, peony extract, Calamus Root extract, white birch extract, Horsetail extract,Hedera Helix(Ivy) extract, hawthorn extract, Sambucus nigra extract, Achillea millefolium extract, Mentha piperita extract, sage extract, mallow extract, Cnidium officinale Root extract, Japanese green gentian extract, soybean extract, jujube extract, thyme extract, tea extract, clove extract, Gramineae imperata cyrillo extract, Citrus unshiu peel extract Japanese Angellica Root extract, Calendula extract, Peach Kernel extract, Bitter orange peel extract, Houttuyna cordata extract, tomato extract, natto extract, Ginseng extract, Green tea extract (camelliea sinesis), garlic extract, wild rose extract, hibiscus extract, Ophiopogon tuber extarct, Nelumbo nucifera extract, parsley extract, honey, hamamelis extract, Parietaria extract, Isodonis herba extract, bisabolol extract, Loquat extract, coltsfoot extract, butterbur extract, Porid cocos wolf extract, extract of butcher's broom, grape extract, propolis extract, luffa extract, safflower extract, peppermintextract, linden tree extract, Paeonia extract, hop extract, pine tree extract, horse chestnut extract, Mizu-bashou [Lysichiton camtschatcese]extract, Mukurossi peel extract, Melissa extract, peach extract, cornflower extract, eucalyptus extract, saxifrage extract, citron extract, coix extract, mugwort extract, lavender extract, apple extract, lettuce extract, lemon extract, Chinese milk vetch extract, rose extract, rosemary extract, Roman Chamomile extract, and royal jelly extract.

The amount of component E) present in the silicone gel composition may vary, but typically range as follows;
0.05 to 50 wt %, alternatively 1 to 25 wt %, or alternatively 1 to 10 wt %, based on the amount by weight of silicone elastomer gel present in the composition, that is total weight of components A), B), C) and D) in the silicone gel composition.

The active, component E), may be added to the silicone gel composition either during the making of the silicone elastomer (pre-load method), or added after the formation of the silicone elastomer gel (post load method).

### The Silicone Elastomer

The silicone elastomers of the present invention are derivable as hydrosilylation reaction products of components A), B), and C). The term “hydrosilylation” means the addition of an organosilicon compound containing silicon-bonded hydrogen, (such as component A) to a compound containing aliphatic unsaturation (such as component B), in the presence of a catalyst (such as component C). Hydrosilylation reactions are known in the art, and any such known methods or techniques may be used to effect the hydrosilylation reaction of components A), B), and C) to prepare the silicone elastomers of the present invention.

The hydrosilylation reaction may be conducted in the presence of a solvent, and the solvent subsequently removed by known techniques. Alternatively, the hydrosilylation may be conducted in a solvent, where the solvent is the same as the carrier fluid described as optional component D).

Alternatively, the silicone elastomers may be prepared by a process comprising:
I) reacting;
   a) an organohydrogencyclosiloxane having at least two SiH units on a siloxane ring,
   B) a compound containing at least two aliphatic unsaturated groups in its molecules,
   C) a hydrosilylation catalyst
      to form
   A) an organohydrogensiloxane having at least two SiH containing cyclosiloxane rings in its molecule,
      wherein the molar ratio of the SiH units of component a) to the aliphatic unsaturated groups of component B) ranges from 2/1 to 8/1,
      alternatively from 2/1 to 6/1,
         or alternatively from 3/1 to 4/1,
II) further reacting;
   A) the organohydrogensiloxane having at least two SiH containing cyclosiloxane rings in its molecule, with additional quantities of
   B) the compound containing at least two aliphatic unsaturated groups in its molecules,
   C) the hydrosilylation catalyst.
      to form a silicone elastomer.

Components a, A), B), C) are the same as those described above. Also, the reaction may be conducted under similar conditions as described above. In aforementioned step II) the molar ratio of the SiH units of component A) to the aliphatic unsaturated groups of component B) ranges from 10/1 to 1/10,
alternatively from 5/1 to 1/5,
   or alternatively from 4/1 to 1/4,

### Gelled Compositions Containing the Silicone Elastomer

The silicone elastomers can be added to a carrier fluid (as described above as component D) to form gelled compositions, or alternatively be prepared first in a separate reaction and then added to the carrier fluid to obtain a gel. The gelled compositions of the present invention may be characterized by their hardness or firmness. Useful tests to characterize the gels are those recommended by the Gelatin Manufacturers Institute of America such as the use of a “Texture Analyzer” (model TA.XT2, Stable Micro Systems, Inc., Godalming, England). The gel sample is subject to a compression test with the Texture Analyzer having a probe with a 5.0 kg load cell. The probe approaches the surface of the gel at a speed of 0.5 mm/sec and continues compression into the gel to a distance of 5.0 mm, then holds for 1 second before retreating. The Texture Analyzer detects the resistance force the probe experiences during the compression test. The force exhibited by the load cell is plotted as a function of time.

The hardness of the silicone elastomers, gels and elastomer blends (SEBs) for purposes of this invention is defined as the resistance force detected by the probe of the “Texture Analyzer” during the compression test. Two data may used to characterize hardness: Force 1, the force at the maximum compression point (i.e. the 5.0 mm compression point into the gel surface), and Area F-T: the area-force integration during the 1 second hold at the maximum compression point. The average of a total of 5 tests are typically performed for each gel.

The value obtained for Force 1 is converted into Newton (N), by dividing the gram force value by 101.97. (i.e. 1 Newton equals 101.97 g force based on the size of the probe used in this instrument). The second property reported by Texture Analyzer measurement is Area F-T 1:2, in g force·sec. This is the area integration of the force vs. test time cure. This property is indicative of a gel network since it indicates ability to sustain resistance to the compression force, which is relevant to elastomers and gels. The value is reported in g force·sec, and is converted to Newton·sec in SI unit by dividing the value in g force·sec by 101.97.

### Gel Paste Compositions Containing the Silicone Elastomer

The gelled compositions of the present invention can be used to prepare gel paste compositions containing actives by;
I) shearing the silicone elastomer gel, as described above,
II) combining the sheared silicone elastomer gel with additional quantities of
   D) the carrier fluid, as described above, and
   E) a personal or health care active
      to form a gel paste composition.

The silicone elastomer gel compositions of the present invention blends may be considered as discrete crosslinked silicone elastomer gel particles dispersed in carrier fluids. Thus, the silicone elastomer compositions are effective rheological thickeners for lower molecular weight silicone fluids. As such they can be used to prepare useful gel blend compositions, such as “paste” compositions.

To make such silicone elastomer blends, the aforementioned silicone elastomer gels of known initial elastomer content (IEC) are sheared to obtain small particle size and further diluted to a final elastomer content (FEC). “Shearing”, as used herein refers to any shear mixing process, such as obtained from homogenizing, sonalating, or any other mixing processes known in the art as shear mixing. The shear mixing of the silicone elastomer gel composition results in a composition having reduced particle size. The subsequent composition having reduced particle size is then further combined with D) the carrier fluid. The carrier fluid may be any carrier fluid as described above, but typically is a volatile methyl siloxane, such as D5. The technique for combining the D) the carrier fluid with the silicone elastomer composition having reduced particle size is not critical, and typically involves simple stirring or mixing. The resulting compositions may be considered as a paste, having a viscosity greater than 100,000 cP (mPa·s).

### F) Additional Optional Components

Composition according to the invention may also contain a number of optional ingredients. In particular, these optional components are selected from those known in the state of the art to be ingredient in personal care formulations. Illustrative, non-limiting examples include; surfactants, solvents, powders, coloring agents, thickeners, waxes, stabilizing agents, pH regulators, and silicones.

Thickening agent may be added to provide a convenient viscosity. For example, viscosities within the range of 500 to 25,000 mm²/s at 25° C. or more alternatively in the range of 3,000 to 7,000 mm²/s are usually suitable. Suitable thickening agents are exemplified by sodium alginate, gum arabic, polyoxyethylene, guar gum, hydroxypropyl guar gum, ethoxylated alcohols, such as laureth-4 or polyethylene glycol 400, cellulose derivatives exemplified by methylcellulose, methylhydroxypropylcellulose, hydroxypropylcellulose, polypropylhydroxyethylcellulose, starch, and starch derivatives exemplified by hydroxyethylamylose and starch amylose, locust bean gum, electrolytes exemplified by sodium chloride and ammonium chloride, and saccharides such as fructose and glucose, and derivatives of saccharides such as PEG-120 methyl glucose diolate or mixtures of 2 or more of these. Alternatively the thickening agent is selected from cellulose derivatives, saccharide derivatives, and electrolytes, or from a combination of two or more of the above thickening agents exemplified by a combination of a cellulose derivative and any electrolyte, and a starch derivative and any electrolyte. The thickening agent, where used is present in the shampoo compositions of this invention in an amount sufficient to provide a viscosity in the final shampoo composition of from 500 to 25,000 mm²/s. Alternatively the thickening agent is present in an amount from about 0.05 to 10 wt % and alternatively 0.05 to 5 wt % based on the total weight of the composition.

Stabilizing agents can be used in the water phase of the compositions. Suitable water phase stabilizing agents can include alone or in combination one or more electrolytes, polyols, alcohols such as ethyl alcohol, and hydrocolloids. Typical electrolytes are alkali metal salts and alkaline earth salts, especially the chloride, borate, citrate, and sulfate salts of sodium, potassium, calcium and magnesium, as well as aluminum chlorohydrate, and polyelectrolytes, especially hyaluronic acid and sodium hyaluronate. When the stabilizing agent is, or includes, an electrolyte, it amounts to about 0.1 to 5 wt % and more alternatively 0.5 to 3 wt % of the total composition. The hydrocolloids include gums, such as Xantham gum or Veegum and thickening agents, such as carboxymethyl cellulose. Polyols, such as glycerine, glycols, and sorbitols can also be used. Alternative polyols are glycerine, propylene glycol, sorbitol and butylene glycol. If a large amount of a polyol is used, one need not add the electrolyte. However, it is typical to use a combination of an electrolyte, a polyol and an hydrocolloid to stabilize the water phase, e.g. magnesium sulfate, butylene glycol and Xantham gum.

Other additives can include powders and pigments especially when the composition according to the invention is intended to be used for make-up. The powder component of the invention can be generally defined as dry, particulate matter having a particle size of 0.02-50 microns. The particulate matter may be colored or non-colored (for example white). Suitable powders include but not limited to bismuth oxychloride, titanated mica, fumed silica, spherical silica beads, polymethylmethacrylate beads, boron nitride, aluminum silicate, aluminum starch octenylsuccinate, bentonite, kaolin, magnesium aluminum silicate, silica, talc, mica, titanium dioxide, kaolin, nylon, silk powder. The above mentioned powders may be surface treated to render the particles hydrophobic in nature.

The powder component also comprises various organic and inorganic pigments. The organic pigments are generally various aromatic types including azo, indigoid, triphenylmethane, anthraquinone, and xanthine dyes which are designated as D&C and FD&C blues, browns, greens, oranges, reds, yellows, etc. Inorganic pigments generally consist of insoluble metallic salts of certified color additives, referred to as the Lakes or iron oxides.A pulverulent colouring agent, such as carbon black, chromium or iron oxides, ultramarines, manganese pyrophosphate, iron blue, and titanium dioxide, pearlescent agents, generally used as a mixture with coloured pigments, or some organic dyes, generally used as a mixture with coloured pigments and commonly used in the cosmetics industry, can be added to the composition. In general, these coulouring agents can be present in an amount by weight from 0 to 20% with respect to the weight of the final composition.
Pulverulent inorganic or organic fillers can also be added, generally in an amount by weight from 0 to 40% with respect to the weight of the final composition. These pulverulent fillers can be chosen from talc, micas, kaolin, zinc or titanium oxides, calcium or magnesium carbonates, silica, spherical titanium dioxide, glass or ceramic beads, metal soaps derived from carboxylic acids having 8-22 carbon atoms, non-expanded synthetic polymer powders, expanded powders and powders from natural organic compounds, such as cereal starches, which may or may not be crosslinked, copolymer microspheres such as EXPANCEL (Nobel Industrie), polytrap and silicone resin microbeads (TOSPEARL from Toshiba, for example).

The waxes or wax-like materials useful in the composition according of the invention have generally have a melting point range of 35 to120° C. at atmospheric pressure. Waxes in this category include synthetic wax, ceresin, paraffin, ozokerite, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, or mixtures thereof. Mention may be made, among the waxes capable of being used as non-silicone fatty substances, of animal waxes, such as beeswax; vegetable waxes, such as carnauba, candelilla wax ; mineral waxes, for example paraffin or lignite wax or microcrystalline waxes or ozokerites; synthetic waxes, including polyethylene waxes, and waxes obtained by the Fischer-Tropsch synthesis. Mention may be made, among the silicone waxes, of polymethylsiloxane alkyls, alkoxys and/or esters.

Such optional components include other silicones (including any already described above), organofunctional siloxanes, alkylmethylsiloxanes, siloxane resins and silicone gums.

Alkylmethylsiloxanes may be included in the present compositions. These siloxane polymers generally will have the formula Me₃SiO[Me₂SiO]_{y}[MeRSiO]_{z}SiMe₃, in which R is a hydrocarbon group containing 6-30 carbon atoms, Me represents methyl, and the degree of polymerization (DP), i.e., the sum of y and z is 3-50. Both the volatile and liquid species of alkymethysiloxanes can be used in the composition.

Silicone gums may be included in the present compositions. Polydiorganosiloxane gums are known in the art and are available commercially. They consist of generally insoluble polydiorganosiloxanes having a viscosity in excess of 1,000,000 centistoke (mm²/s) at 25° C., alternatively greater than 5,000,000 centistoke (mm²/s) at 25° C. These silicone gums are typically sold as compositions already dispersed in a suitable solvent to facilitate their handling. Ultra-high viscosity silicones can also be included as optional ingredients. These ultra-high viscosity silicones typically have a kinematic viscosity greater than 5 million centistoke (mm²/s) at 25° C., to about 20 million centistoke (mm²/s) at 25° C. Compositions of this type in the form of suspensions are most preferred, and are described for example in U.S. Pat. No. 6,013,682 (Jan. 11, 2000).

Silicone resins may be included in the present compositions. These resin compositions are generally highly crosslinked polymeric siloxanes. Crosslinking is obtained by incorporating trifunctional and/or tetrafunctional silanes with the monofunctional silane and/or difunctional silane monomers used during manufacture. The degree of crosslinking required to obtain a suitable silicone resin will vary according to the specifics of the silane monomer units incorporated during manufacture of the silicone resin. In general, any silicone having a sufficient level of trifunctional and tetrafunctional siloxane monomer units, and hence possessing sufficient levels of crosslinking to dry down to a rigid or a hard film can be considered to be suitable for use as the silicone resin. Commercially available silicone resins suitable for applications herein are generally supplied in an unhardened form in low viscosity volatile or nonvolatile silicone fluids. The silicone resins should be incorporated into compositions of the invention in their non-hardened forms rather than as hardened resinous structures.

Silicone carbinol Fluids may be included in the present compositions. These materials are described in WO 03/101412 A2, and can be commonly described as substituted hydrocarbyl functional siloxane fluids or resins.

Water soluble or water dispersible silicone polyether compositions may be included in the present compositions: These are also known as polyalkylene oxide silicone copolymers, silicone poly(oxyalkylene) copolymers, silicone glycol copolymers, or silicone surfactants. These can be linear rake or graft type materials, or ABA type where the B is the siloxane polymer block, and the A is the poly(oxyalkylene) group. The poly(oxyalkylene) group can consist of polyethylene oxide, polypropylene oxide, or mixed polyethylene oxide/polypropylene oxide groups. Other oxides, such as butylene oxide or phenylene oxide are also possible.

Compositions according to the invention can be used in w/o, w/s, or multiple phase emulsions using silicone emulsifiers. Typically the water-in-silicone emulsifier in such formulation is non-ionic and is selected from polyoxyalkylene-substituted silicones, silicone alkanolamides, silicone esters and silicone glycosides. Suitable silicone-based surfactants are well known in the art, and have been described for example in U.S. Pat. No. 4,122,029 (Gee et al.), U.S. Pat. No. 5,387,417 (Rentsch), and U.S. Pat. No. 5,811,487 (Schulz et al).

When the composition according to the invention is an oil-in-water emulsion, it will include common ingredients generally used for preparing emulsions such as but not limited to non ionic surfactants well known in the art to prepare o/w emulsions. Examples of nonionic surfactants include polyoxyethylene alkyl ethers, polyoxyethylene alkylphenol ethers, polyoxyethylene lauryl ethers, polyoxyethylene sorbitan monoleates, polyoxyethylene alkyl esters, polyoxyethylene sorbitan alkyl esters, polyethylene glycol, polypropylene glycol, diethylene glycol, ethoxylated trimethylnonanols, and polyoxyalkylene glycol modified polysiloxane surfactants.

The composition according to the invention can also be under the form of aerosols in combination with propellant gases, such as carbon dioxide, nitrogen, nitrous oxide, volatile hydrocarbons such as butane, isobutane, or propane and chlorinated or fluorinated hydrocarbons such as dichlorodifluoromethane and dichlorotetrafluoroethane or dimethylether.

The silicone elastomer gel compositions can be used in a variety of personal, household, and healthcare applications. In particular, the compositions of the present invention may be used: as thickening agents, as taught in U.S. Pat. Nos. 6,051,216, 5,919,441, 5,981,680; to structure oils, as disclosed in WO 2004/060271 and WO 2004/060101; in sunscreen compositions as taught in WO 2004/060276; as structuring agents in cosmetic compositions also containing film-forming resins, as disclosed in WO 03/105801; in the cosmetic compositions as taught in US Patent Application Publications 2003/0235553, 2003/0072730, 2003/0170188, EP 1,266,647, EP 1,266,648, EP1,266,653, WO 03/105789, WO 2004/000247 and WO 03/106614; as structuring agents as taught in WO 2004/054523; in long wearing cosmetic compositions as taught in US Patent Application Publication 2004/0180032; in transparent or translucent care and/or make up compositions as discussed in WO 2004/054524; all of which are incorporated herein by reference.

Silicone elastomer gels can also be used in anti-perspirant and deodorant compositions under but not limited to the form of sticks, soft solid, roll on, aerosol, and pumpsprays. Some examples of antiperspirant agents and deodorant agents are Aluminum Chloride, Aluminum Zirconium Tetrachlorohydrex GLY, Aluminum Zirconium Tetrachlorohydrex PEG, Aluminum Chlorohydrex, Aluminum Zirconium Tetrachlorohydrex PG, Aluminum Chlorohydrex PEG, Aluminum Zirconium Trichlorohydrate, Aluminum Chlorohydrex PG, Aluminum Zirconium Trichlorohydrex GLY, Hexachlorophene,

Chloride, Aluminum Sesquichlorohydrate, Sodium Bicarbonate, Aluminum Sesquichlorohydrex PEG, Chlorophyllin-Copper Complex, Triclosan, Aluminum Zirconium Octachlorohydrate, and Zinc Ricinoleate.

The personal care compositions of this invention may be in the form of a cream, a gel, a powder, a paste, or a freely pourable liquid. Generally, such compositions can generally be prepared at room temperature if no solid materials at room temperature are presents in the compositions, using simple propeller mixers, Brookfield counter-rotating mixers, or homogenizing mixers. No special equipment or processing conditions are typically required. Depending on the type of form made, the method of preparation will be different, but such methods are well known in the art.

The compositions according to this invention can be used by the standard methods, such as applying them to the human body, e.g. skin or hair, using applicators, brushes, applying by hand, pouring them and/or possibly rubbing or massaging the composition onto or into the body. Removal methods, for example for colour cosmetics are also well known standard methods, including washing, wiping, peeling and the like. For use on the skin, the compositions according to the present invention may be used in a conventional manner for example for conditioning the skin. An effective amount of the composition for the purpose is applied to the skin. Such effective amounts generally range from about 1mg/cm² to about 3 mg/cm². Application to the skin typically includes working the composition into the skin. This method for applying to the skin comprises the steps of contacting the skin with the composition in an effective amount and then rubbing the composition into the skin. These steps can be repeated as many times as desired to achieve the desired benefit.

The use of the compositions according to the invention on hair may use a conventional manner for conditioning hair. An effective amount of the composition for conditioning hair is applied to the hair. Such effective amounts generally range from about lg to about 50 g, preferably from about 1 g to about 20 g. Application to the hair typically includes working the composition through the hair such that most or all of the hair is contacted with the composition. This method for conditioning the hair comprises the steps of applying an effective amount of the hair care composition to the hair, and then working the composition through the hair. These steps can be repeated as many times as desired to achieve the desired conditioning benefit. When a high silicone content is incorporated in a hair care composition according to the invention, this may be a useful material for split end hair products.

The compositions according to this invention can be used on the skin of humans or animals for example to moisturize, color or generally improve the appearance or to apply actives, such as sunscreens, deodorants, insect repellents etc.

### EXAMPLES

These examples are intended to illustrate the invention to one of ordinary skill in the art and are should not be interpreted as limiting the scope of the invention set forth in the claims.

### Materials Description

The following materials were used in these examples.

### Carrier Fluids

D5=decamethylcyclopentasiloxane or D5 cyclics, DC245 (Dow Corning Corporation, Midland Mich.) used as provided.
IDNP=isodecyl neopentanoate obtained from ISP (International Specialty Products Co) under the trade name of CERAPHYL SLK. ps IDD=ISODODECANE (Permethyl 99A from Presperse Incorporated, Somerset, N.J.)

### Methods of Measuring Viscosity of Silicone Elastomer Blends (SEBs)

The Brookfield Helipath™ Stand, when used with a suitable Brookfield Viscometer fitted with a special T-bar type spindle, will permit viscosity/consistency measurements in centipoise values for materials having characteristics similar to paste, putty, cream, gelatin, or wax.

The viscosity of silicone elastomer blends was determined using a Brookfield Model RVD-II+ Viscometer with Helipath stand (Brookfield Model D) and T-Bar spindles (Brookfield Helipath Spindle Set). All were purchased from Brookfield Engineering Laboratories, Inc. (11 Commerce Boulevard Middleboro, Mass., USA).

A sample size of 100 g in a 4 oz. round jar was required. The following preparation procedure was used before measurement: the sample was de-aired first via centrifuge, then vacuum de-aired for two hours. After de-airing, the sample was conditioned for a minimum of 4 hours@25° C. The sample was positioned with T-bar spindle at center. The reading was taken according to the typical procedure for Helipath spindle.

In general, spindle 93 (T-bar spindle C) is used for the less viscous sample, spindle 95 (T-bar spindle E) for the more viscous samples. The standard setting for rpm was 2.5. The spindle speed is maintained at constant 2.5 rpm and spindle was varied to handle samples with significant viscosities.

### Measurement of Silicone Elastomer Gel Hardness

The hardness (or firmness) of silicone elastomer gels was characterized using a Texture analyzer (model TA.XT2, Stable Micro Systems, Inc., Godalming, England). The Gelatin Manufacturers Institute of America recommends such test methods as a standard procedure.

For silicone gels and elastomer blends, ½inch (1.27 cm) diameter cylindrical probe made of DELRIN acetal resin (Dupont) was used for the measurement. The gel sample is subject to the compression test using the probe with the following test cycle: the probe approaches the surface of the gel at a speed of 0.5 mm/sec and continues compression into the gel to a distance of 5.0 mm, then holds for 1 second before retreating. The Texture Analyzer has a 5.0 Kg load cell to detect the resistance force the probe experiences during the compression test. The force exhibited by the load cell is plotted as a function of time.

The hardness of the silicone elastomers, gels and elastomer blends (SEBs) is defined as the resistance force detected by the probe during the compression test. Two data are used for the hardness value: Force 1: the force at the maximum compression point (i.e. the 5.0 mm compression point into the gel surface), and Area F-T: the area-force integration during the 1 second hold at the maximum compression point. A total of 5 tests were performed for each gel and the average of the five tests is reported.

Texture Analyzer used for gel hardness measurement is force in gram, as detected by the transducer. Two values are reported for gel hardness: Force 1, the force in gram registered when the probe reached its pre-programmed full indentation (or compression) in gel sample. The unit for Force 1 reading is gram force.

The value obtained for Force 1 is converted into Newton (N), by dividing the gram force value by 101.97. (i.e. 1 Newton equals 101.97 g force based on the size of the probe used in this instrument). For instance, a value of 6327 g force converts to 62.0 N.

The second property reported by Texture Analyzer measurement is Area F-T 1:2, in g force·sec. This is the area integration of the force vs. test time cure. This is an indicative property of a gel network as it indicates it ability to sustain resistance to the compression force, which is relevant to elastomers and gels.

The value is reported in g force·sec, and is converted to Newton·sec in SI unit by dividing the value in g force·sec by 101.97. For instance, a value of 33,947 g force·sec is 332.9 N·s in SI units.

TABLE 1
Description of Elastomers

| | Silicone Elastomer Blend (SEB) Reference | | | | |
|---|---|---|---|---|---|
| | Prior art | | | | |
| | SEB¹ | SEB A² | SEB B² | SEB C² | SEB D³ |
| Description | Silicone | Silicone | Silicone | Silicone | Silicone |
| | elastomer | elastomer | elastomer | elastomer | Polyether |
| | in IDD | in IDD | in IDD | in IDD | elastomer |
| | | | | | in IDD |
| Wt. % FEC in SEB | 18% | 11.0 | 11.0 | 11.0 | 11.0 |
| Carrier fluid | IDD | IDD | IDD | IDD | IDD |
| Viscosity (cps) | 431,000 | 134,889 | 365,000 | 353,111 | 215,222 |
| SiH compound | Linear rake | Cyclic SiH | Cyclic SiH | Cyclic SiH | Cyclic SiH |
| | type | derived⁵ | derived⁵ | derived⁵ | derived⁵ |
| | MDₓD^{H}_{y}M | | | | |
| Vinyl | 1,5- | M^{vi}DₓM^{vi} | M^{vi}DₓM^{vi} | M^{vi}DₓM^{vi} | Bis-allyl |
| compound | hexadiene | (37dp) | (100dp) | (130dp) | PO20/ |
| | | | | | M^{vi}DₓM^{vi} |
| | | | | | (130dp) |
| % Organic in Gel | | 0 | 0 | 0 | 20 |
| IEC % in starting gel | 18 | 20 | 20 | 20 | 20 |

Silicone Elastomer Blend (SEB) Reference

| | SEB E³ | SEB G³ | SEB H⁴ | SEB I⁴ |
|---|---|---|---|---|
| Description | Silicone | Silicone | Silicone | Silicone |
| | Polyether | Polyether | Organic | Organic |
| | elastomer | elastomer | elastomer | elastomer |
| | in IDD | in IDD | in IDD | in IDD |
| Wt. % FEC in SEB | 11.0 | 11.0 | 12.0 | 12.0 |
| Carrier fluid | IDD | IDD | IDD | IDD |
| Viscosity (cps) | 131,333 | 126,667 | 389,056 | 270,444 |
| SiH compound | Cyclic SiH | Cyclic SiH | Cyclic SiH | Cyclic SiH |
| | derived⁵ | derived⁵ | derived⁶ | derived⁶ |
| Vinyl | Bis-allyl | Bis-allyl | Bis-hexenyl | Bis-hexenyl |
| compound | PO20 | PO50/ | C48⁷/ | C48⁷ |
| | | M^{vi}DₓM^{vi} | M^{vi}DₓM^{vi} | |
| | | (130dp) | (130dp) | |
| % Organic in Gel | 28.5 | 40 | 20 | 40 |
| IEC % in starting gel | 20 | 20 | 20 | 20 |

¹A silicone elastomer gel in IDD (from Dow Corning Corporation, Midland MI), representative of the silicone elastomers taught in U.S. Pat. No. 5,880,210.
²A silicone elastomer gel representative of those described in US 60/784,340 and US 60/838,803.
³A silicone elastomer gel representative of those described in US 60/799,864 and US 60/838,802.
⁴A silicone elastomer gel representative of those described in US 60/849,397 and US 60/874,203.
⁵see Example 1C of US 60/799,864.
⁶see Example 1A of US 60/874,203.
⁷see Example 3A of US 60/849,397.

The threshold for each product is the lowest level of product diluted in isododecane for which the panelists feel a significant difference in comparison with pure Isododecane. The perception threshold of the different materials was evaluated with Triangular sensory tests.

The aim of this test is to determine the level of elastomer blend that one has to incorporate in a formulation to induce an effect on the sensory, and to compare this level to the level of the Prior art SEB.

The perception threshold level was exactly determined for Prior Art SEB and SEB C. The other products perception threshold levels were evaluated as being higher or lower than the Prior Art SEB.

Conditions of the test:
a maximum of 18 panelists is required for a comparison of two products
products are randomly allocated on three sites of the forearm: 2 sites with a same treatment and one with a different treatment
a significant difference at 99% of confidence level exists between the two products when 12 among the 18 panelists have picked up the tress with the different treatment
as soon as 7 panelists failed the test, the evaluation can be stopped

| Prior art | | | | | |
|---|---|---|---|---|---|
| SEB | SEB C | SEB A | SEB D | SEB E | SEB H |
| 23% | 26% | >23% | <23% | <23% | <23% |

The Prior art SEB has been pre-diluted to 12% before evaluation. Generally, the SEB's listed in Table 1 have lower threshold levels than the Prior art SEB. Thus, they can be incorporated in formulations at a lower level than Prior art SEB while bringing the same sensory benefits.

The Silicone Elastomer Blends, as described in Table 1 were individually formulated in different personal care formulations, as shown by the following examples.

### Color Cosmetics

### Castor Oil-Based Lipstick Formulation

TABLE 2
Castor oil-based Lipstick Formulation

| Raw Material | INCI Name | Wt % |
|---|---|---|
| Phase A | | |
| castor oil | | 43.7 |
| Softisan 100 | Hydrogenated coco-glycerides | 8 |
| cerilla G | Candelilla Cera | 9 |
| Softisan 645 | Bis-Diglyderyl Polyacyladipate | 8 |
| Cerabeil Blanchie DAB | Cera alba | 3 |
| Cerauba T1 | Cera Carnauba | 2 |
| Trivent OC-G | Ticaprylin | 15 |
| Vitamin E acetate | Tocopheryl acetate | 0.5 |
| Propyl Paraben | | 0.1 |
| BHT(2,6-di-tert-butyl-4-methylphenol) | | 0.05 |
| A Silicone Elastomer | | 5 |
| Blend of Table 1 | | |
| Total phase A | | 94.3 |
| Phase B | | |
| COD 8008 | White | 1 |
| COD 8005 | Yellow | 3 |
| COD 8006 | Red | 1.7 |
| Total Phase B | | 5.7 |

Procedure:
1. Heat Phase A to 85 C.
2. Add Phase B.
3. Pour into lipstick molds.
4. Place in freezer for 60 min.
5. Remove from molds.

### Cyclopentasiloxane-Based Lipstick Formulation

TABLE 3
Cyclopentasiloxane-based Lipstick
Formulation with 5% Elastomer Blend

| Raw Material | INCI Name | % |
|---|---|---|
| Phase A | | |
| White ozokerite wax | | 4 |
| Cerilla G | candellila wax | 11 |
| Eutanol G | Octyl dodecanol | 25 |
| Dow Corning 245 | Cyclopentasiloxane | 5 |
| Silicone Elastomer | | 5 |
| Blend of Table 1 | | |
| Petrolatum | vaselinum | 4 |
| Fluilan | lanolin oil | 9 |
| Avocado oil | | 2 |
| Novol | Oleyl alcohol | 8 |
| pigment blend | | 27 |
| | | 100 |
| Pigment blend | | |
| Covasil TiO2 | | 5 |
| Dow Corning 245 | Cyclopentasiloxane | 77.5 |
| Covasil red W3801 | | 17.5 |
| | | 100 |

Procedure:
1. Heat phase A to 85 C except pigment blend.
2. Add pigment blend.
3. Pour formulation into lipstick mold.
4. Place in freezer for 60 min.
5. Remove from molds

### Stick Formulation

TABLE 4
Stick Formulation

| Raw Material | | % |
|---|---|---|
| Phase A | | |
| Unipure Red LC 304 AS | CI 15850 and Triethoxycaprylylsilane | 6.4 |
| Unipure Black LC 989 AS-EM | CI 77499 and Triethoxycaprylylsilane | 0.8 |
| Dow Corning ® PH-1555 HRI | Trimethyl Pentaphenyl Trisiloxane | 14.5 |
| COSMETIC FLUID | | |
| Phase B | | |
| Dow Corning ® AMS-C30 COSMETIC WAX | C30-45 Alkyl Methicone (and) C30- | 6.8 |
| | 45 Olefin | |
| Dow Corning ® 2503 COSMETIC WAX | Stearyl Dimethicone | 2.2 |
| Covalip LL 48 | Ozokerite and Euphorbia Cerifera | 11.5 |
| | (Candelilla) Wax and Isostearyl | |
| | Alcohol and Isopropyl Palmitate and | |
| | Myristyl Lactate and Synthetic | |
| | Beeswax and Copernicia Cerifera | |
| | (Carnauba) Wax and Quaternium-18 | |
| | Hectorite and Propylene Carbonate and | |
| | Ethylene/VA Copolymer and | |
| | Propylparaben | |
| Covasterol | Glyceryl Isostearate and Isostearyl | 0.5 |
| | Alcohol and Beta-Sitosterol and | |
| | Butyrospermum Parkii (Shea Butter) | |
| | and Euphorbia Cerifera (Candelilla) | |
| | Wax | |
| Phase C | | |
| Covapearl Satin 931 AS | Mica and CI 77891 and | 10 |
| | Triethoxycaprylylsilane | |
| Covafluid FS | Sodium Stearyl Fumarate | 1 |
| Phase D | | |
| Dow Corning ® 245 FLUID | Cyclopentasiloxane | 18 |
| Silicone Elastomer Blend of Table 1 | | 27.8 |

Procedure:
1. Grind pigment of phase A in silicone with high shear mixer
2. Mix ingredients of phase B and heat to 80° C.
3. Add phase A to phase B while stirring and continued heating
4. Add phase C with mixing
5. Add phase D under stirring and maintain the temperature at 70° C.
6. Pour in the mould at 70° C.

### Liquid Lipstick: Long Lasting

TABLE 5
Liquid Lipstick: Long lasting

| Raw Material | % |
|---|---|
| Phase A | |
| Unipure Red LC 304 AS/LCW Sensient | 3.6 |
| Unipure Red LC 3075 AS/LCW Sensient | 3.6 |
| Isododecane | 10 |
| Phase B | |
| Dow Corning ® AMS-C30 COSMETIC WAX | 6 |
| Isododecane | 42.2 |
| Phase C | |
| Silicone Elastomer Blend of Table 1 | 26.4 |
| Phase D | |
| Paragon MEPB | 0.2 |
| Phase E | |
| Covapearl Rich Gold 230 AS/LCW Sensient | 8 |

Procedure:
1. Mix phase A ingredients together
2. Homogenize using a high shear mixer (Ultraturrax or Silverson type)
3. Heat Ingredient 4 to 80° C.
4. Tare the beaker (final beaker), warm the isododecane up to 40° C. covering the beaker with an aluminium foil
5. Add ingredient 4 to 5, stop heating and ensure a homogeneous mixture
6. Add phase A to phase B with gentle mixing
7. Add phase C with mixing
8. Add phase D with slow mixing
9. Add phase E with slow mixing
10. Finally compensate the solvent loss with isododecane

### Foundation Cream

TABLE 6
Foundation Cream with elastomer blend

| Raw Material | | % |
|---|---|---|
| Phase A | | |
| DC 2-1184 | | 11.0% |
| TiO2 W877 | Titanium dioxide | 11.0% |
| Yellow W 1802 | Iron Oxide | 2.5% |
| Red W 3801 | Iron Oxide | 1.5% |
| Black W 9801 | Iron Oxide | 0.6% |
| Phase B | | |
| Silicone Elastomer | | 12.0% |
| Blend of Table 1 | | |
| Sepicide HB | Parabens blend | 0.5% |
| Dow Corning ® 5200 | Cyclomethicone (and) PEG/PPG- | 2.0% |
| | 18/18 Dimethicone | |
| Phase C | | |
| Tween 20 | Polysorbate 20 | 0.5% |
| NaCl | | 1.0% |
| Distilled water | | 57.4% |

Procedure:
1. Mix ingredients of Phase A and homogenize using a high shear mixer
2. Add Elastomer Blend, when melted, add remain of Phase B
3. Mix ingredients of Phase C in another beaker
4. Add Phase C very slowly into Phase A+B under agitation (1200 rpm)
5. When addition is completed, leave under agitation for an additional 5 minutes and pass through a homogenizer

### Tinted Sunscreen

TABLE 7
Tinted sunscreen

| Phase A | | |
|---|---|---|
| Dow Corning ® 5200 | | 2.0 |
| FORMULATION AID | | |
| Silicone Elastomer | | 20.0 |
| Blend of Table 1 | | |
| Dow Corning ® 556 FLUID | | 3.0 |
| Parsol MCX | Octyl Methoxycinnamate | 6.0 |
| Phase B | | |
| Pigment blend | | 20 |
| Phase C | | |
| Deionized water | | 47.4 |
| Disodium EDTA | | 0.2 |
| Sodium Chloride | | 1 |
| Nipaguard DMDH | | 0.2 |
| Polysorbate 20 | Tween 20 | 0.2 |
| | Pigment blend | |
| 2-1184 | | |

Procedure:
1. Mix phase A ingredients together
2. Grind Phase B ingredients together and add to phase A with mixing
3. Mix phase C ingredients together
4. Check pH of water phase (5.5-6.5) and correct if necessary
5. Add phase C to AB blend, slowly and with turbulent mixing (about 1000 rpm)
6. Continue mixing for 10 minutes at the same speed
7. Pass the mixture through high shear device to get uniform particle size distribution

### Shower Gels

### Shower Gel: Smooth After Feel

TABLE 8
Shower Gel: Smooth after Feel (CPF 162)

| Raw Material | Commercial name | % |
|---|---|---|
| Phase A | | |
| Distilled Water | | 57.37 |
| Crothix 2602 | | 0.5 |
| Propylene Gycol | | 1.0 |
| Phase B | | |
| Sodium Laureth Sulfate | Empicol ESB 3 | 20 |
| Ammonium Laureth Sulfate | Empicol EAC 70 | 6.43 |
| Cocamidopropyl betaine | Amonyl 380 BA | 8.0 |
| Cocamide MIPA | Ninol M-10 | 4.0 |
| Silicone Elastomer | | 2.0 |
| Blend of Table 1 | | |
| Phase C | | |
| Nipaguard DMDH | | 0.2 |
| Phase D | | |
| Citric acid 50% | | 0.5 |

Procedure:
1. Mix ingredients of Phase A
2. Mix Phase B ingredients together and add to Phase A with mixing
3. Add Phase C and mix
4. Adjust the pH with phase D at 5.5-6
5. Pour into containers

1. A personal care composition comprising a silicone elastomer gel containing a silicone elastomer derived from;
A) an organohydrogensiloxane having at least two SiH containing cyclosiloxane rings in its molecule
B) a compound having at least two aliphatic unsaturated groups in its molecule,
C) a hydrosilylation catalyst,
D) an optional carrier fluid;
E) an optional personal care or healthcare active.

2. The composition of claim 1 wherein the silicone gel composition has a hardness of at least 0.03 Newton force.

3. The composition of claim 1 wherein the organohydrogensiloxane has the formula G-[Y-G]ₐ where G is a cyclosiloxane containing at least one SiH unit and Y is a divalent organic group, a siloxane group, a polyoxyalkylene group, polyalkylene, a hydrocarbon-silicone copolymer, or combination thereof, and a is greater than zero.

4. The composition of claim 1 wherein the organohydrogensiloxane is prepared by a hydrosilylation reaction of
a) an organohydrogencyclosiloxane having at least two SiH units on the siloxane ring and,
B) a compound containing at least two aliphatic unsaturated groups in its molecule, wherein the molar ratio of SiH units to unsaturated group ranges from 2/1 to 8/1.

5. The composition of claim 4 wherein the organohydrogencyclosiloxane has the formula
[(CH₃)HSiO]ₘ where m is 3-8.

6. The composition of claim 1 wherein B) the compound containing at least two aliphatic unsaturated groups in its molecule is selected from a compound having the formula R²—Y—R² where R² is a monovalent unsaturated aliphatic group and Y is a divalent hydrocarbon, a siloxane, a polyoxyalkylene, a polyalkylene or polyisoalkylene, a hydrocarbon-silicone copolymer, or mixtures thereof.

7. The composition of claim 1 wherein the compound containing at least two aliphatic unsaturated groups in its molecule is 1,5-hexadiene.

8. The composition of claim 1 wherein the compound containing at least two aliphatic unsaturated groups is selected from an organopolysiloxane comprising at least two siloxane units having a formula R² RₘSiO_{(4-m)/2}
wherein
R is an organic group,
R² is a monovalent unsaturated aliphatic group, and
m is zero to 3.

9. The composition of claim 8 wherein the organopolysiloxane has the formula
(R₂R²SiO_{0.5})(SiO₂)_{w}(R₂R²SiO_{0.5})
(R₂R²SiO_{0.5})(SiO₂)_{w}(R₂SiO)ₓ(R₂R²SiO_{0.5})
(R₂R²SiO_{0.5})(R₂SiO)ₓ(R₂R²SiO_{0.5})
(R₃SiO_{0.5})(R₂SiO)ₓ(R²RSiO)_{y}(R₃SiO_{0.5})
(R₃SiO_{0.5})(R₂SiO)ₓ(R²RSiO)_{y}(RSiO_{1.5})_{z}(R₃SiO_{0.5})
(R₃SiO_{0.5})(R₂SiO)ₓ(R²RSiO)_{y}(SiO₂)_{w}(R₃SiO_{0.5})
where w≧0, x≧0, y≧2, and z is ≧0,
and R is an organic group, R² is a monovalent unsaturated aliphatic group.

10. The composition of claim 9 wherein R is methyl and R² is CH2═CH—.

11. The composition of claim 6 wherein Y in the R²—Y—R² compound is a hydrocarbon-silicone copolymer group having the formula
—[R¹ᵤ(R₂SiO)ᵥ]ₘ—
where R¹ is a divalent hydrocarbon, R is an organic group,
u and v are independently ≧1, and m is >1.

12. The composition of claim 11 wherein
R is methyl,
R¹ is hexylene,
u=1, v and m is >1.

13. The composition of claim 12 wherein v ranges from 2 to 500.

14. The composition of claim 12 where m ranges from 2 to 100.

15. The composition of claim 1 wherein C) the hydrosilylation catalyst is a platinum group containing catalyst.

16. The composition of claim 1 wherein the molar ratio of A)/B) is from 10/1 to 1/10.

17. The composition of claim 1 wherein the carrier fluid is a silicone having a viscosity at 25° C. in the range of 1 to 1,000 mm²/sec.

18. The composition of claim 1 wherein the carrier fluid is decamethylcyclopentasiloxane, isododecane, or isodecyl neopentanoate.

19. The composition of claim 1 wherein E) is a personal care active selected from a vitamin, sunscreen, plant extract, or fragrance.

20. The composition of claim 1 wherein E) is a health care active selected from a topical drug active, protein, enzyme, antifugual, or antimicrobial agent.

21. The composition of claim 1 wherein component E) is vitamin A palmitate.

22. The composition of claim 1 wherein component E) is octyl methoxycinnamate.

23. The composition of claim 1 wherein the personal care composition is selected from a color cosmetic, a lipstick, a foundation, a shampoo, a hair conditioner, a hair fixative, a shower gel, a skin moisturizer, a skin conditioner, a body conditioner, a sun protection product, an antiperspirant, and a deodorant.
